(19) United States
(12) Patent Application Publication
(10) Pub. No.: US 2013/0110174 A1
(43) Pub. Date: May 2, 2013

(54) METHODS FOR INSTALLING A VERTEBRAL CONSTRUCT

(71) Applicant: Gregory C. Marik, Collierville, TN (US)

(72) Inventor: Gregory C. Marik, Collierville, TN (US)

(73) Assignee: Warsaw Orthopedic, Inc., Warsaw, IN (US)

(21) Appl. No.: 13/285,268

(22) Filed: Oct. 31, 2011

(51) Int. Cl. A61B 17/88

(52) U.S. Cl. 606/279

(57) ABSTRACT

A method for installing a vertebral construct includes anchoring a first bone fixation apparatus having a receiver with a channel configured to receive the vertebral construct and a bone anchor configured for engaging a first vertebral body to the first vertebral body. The vertebral construct is positioned within the channel of the receiver of the first bone fixation apparatus. A second bone fixation apparatus having a receiver with a channel configured to receive the vertebral construct and a bone anchor configured for engaging a second vertebral body is anchored into the second vertebral body. The vertebral construct positioned within the channel of the receiver of the first bone fixation apparatus is then positioned within the channel of the receiver of the second bone fixation apparatus so as to secure the vertebral construct to the first and second bone fixation apparatuses.

### TECHNICAL FIELD

The present disclosure generally relates to methods for the treatment of musculoskeletal disorders, and more particularly to methods for installing a vertebral construct.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. As part of these surgical treatments, vertebral constructs such as vertebral rods are often used to provide stability to a treated region. Rods redirect stresses away from a damaged or defective region while healing takes place to restore proper alignment and generally support the vertebral members. During surgical treatment, one or more rods may be attached via one or more bone fasteners to the exterior of one or more vertebral members. Typically, one or more bone fasteners are implanted into the exterior of one or more vertebral members. After one or more bone fasteners are implanted, one or more rods are attached to the one or more bone fasteners. This disclosure describes an improvement over these prior art technologies.

### SUMMARY OF THE INVENTION

Accordingly, methods are provided for installing a vertebral construct. It is contemplated that the methods may be employed in any existing surgical method or technique including open surgery, mini-open surgery, minimally invasive surgery and percutaneous surgical implantation.

In one particular embodiment, in accordance with the principles of the present disclosure, a method for installing a vertebral construct is provided. The method includes anchoring a first bone fixation apparatus having a receiver defined by a pair of opposing arms defining a channel therebetween configured to receive the vertebral construct and a bone anchor having a lower portion configured for engaging a first vertebral body and a head engaged with a lower surface of the receiver into the first vertebral body. The vertebral construct is positioned within the channel of the receiver of the first bone fixation apparatus. A second bone fixation apparatus having a receiver defined by a pair of opposing arms defining a channel therebetween configured to receive the vertebral construct and a bone anchor having a lower portion configured for engaging a second vertebral body and a head engaged with a lower surface of the receiver is anchored into the second vertebral body. The vertebral construct positioned within the channel of the receiver of the first bone fixation apparatus is then positioned within the channel of the receiver of the second bone fixation apparatus, while at least a portion of the vertebral construct remains within the channel of the receiver of the first bone fixation apparatus, so as to secure the vertebral construct to the first and second bone fixation apparatuses.

In one embodiment, the method includes anchoring a first bone fixation apparatus having a receiver defined by a pair of opposing arms defining a channel therebetween configured to receive a vertebral construct and a bone anchor having a lower portion configured for engaging a first vertebral body and a head engaged with a lower surface of the receiver into the first vertebral body. A second bone fixation apparatus having a receiver defined by a pair of opposing arms defining a channel therebetween configured to receive the vertebral construct and a bone anchor having a lower portion configured for engaging a second vertebral body and a head engaged with a lower surface of the receiver is then anchored into the second vertebral body. The vertebral construct is positioned within the channel of the receiver of the first bone fixation apparatus. Next, the vertebral construct positioned within the receiver of the channel of the first bone fixation apparatus is positioned within the channel of the receiver of the second bone fixation apparatus, while at least a portion of the vertebral construct remains positioned within the channel of the receiver of the first bone fixation apparatus. A third bone fixation apparatus having a receiver defined by a pair of opposing arms defining a channel therebetween configured to receive the vertebral construct and a bone anchor having a lower portion configured for engaging a third vertebral body and a head engaged with a lower surface of the receiver is then anchored into the third vertebral body. Next, the vertebral construct positioned within the receivers of the first and second bone fixation apparatuses is positioned within the channel of the receiver of the third bone fixation apparatus, while at least a portion of the vertebral construct remains positioned within the channels of the first and second bone fixation apparatuses, so as to secure the vertebral construct to the first, second and third bone fixation apparatuses.

In one embodiment, the method includes anchoring a first bone fixation apparatus having a receiver defined by a pair of opposing arms defining a channel therebetween configured to receive a vertebral construct and a bone anchor having a lower portion configured for engaging a first vertebral body and a head engaged with a lower surface of the receiver into the first vertebral body. The vertebral construct is positioned within the channel of the receiver of the first bone fixation apparatus. Next, a second bone fixation apparatus having a receiver defined by a pair of opposing arms defining a channel therebetween configured to receive the vertebral construct and a bone anchor having a lower portion configured for engaging a second vertebral body and a head engaged with a lower surface of the receiver is anchored into the second vertebral body. A third bone fixation apparatus having a receiver defined by a pair of opposing arms defining a channel therebetween configured to receive the vertebral construct and a bone anchor having a lower portion configured for engaging a third vertebral body and a head engaged with a lower surface of the receiver is then anchored into the third vertebral body. The vertebral construct positioned within the channel of the receiver of the first bone fixation apparatus is positioned within the channel of the receiver of the second bone fixation apparatus, while at least a portion of the vertebral construct remains positioned within the channel of the receiver of the first bone fixation apparatus. Next, the vertebral construct positioned within the channels of receivers of the first and second bone fixation apparatuses is positioned within the channel of the receiver of the third bone fixation apparatus, while at least a portion of the vertebral construct remains positioned within the channels of the receivers of the first and second bone fixation apparatuses, so as to secure the vertebral construct to the first, second and third bone fixation apparatuses.

### BRIEF DESCRIPTION OF THE DRAWINGS

The present disclosure will become more readily apparent from the specific description accompanied by the following drawings, in which:

FIG. 1A is a side, cross-sectional view illustrating step 1 of one particular embodiment of a method in accordance with the principles of the present disclosure;

FIG. 1B is a side, cross-sectional view illustrating step 2 of a method in accordance with the principles of the present disclosure;

FIG. 1C is a side, cross-sectional view illustrating step 3 of a method in accordance with the principles of the present disclosure;

FIG. 1D is a side, cross-sectional view illustrating step 24 of a method in accordance with the principles of the present disclosure;

FIG. 2A is a side, cross-sectional view illustrating step 1 of a method in accordance with the principles of the present disclosure;

FIG. 2B is a side, cross-sectional view illustrating step 2 of a method in accordance with the principles of the present disclosure;

FIG. 3A is a side, cross-sectional view illustrating step 1 of a method in accordance with the principles of the present disclosure;

FIG. 3B is a side, cross-sectional view illustrating step 2 of a method in accordance with the principles of the present disclosure;

FIG. 3C is a side, cross-sectional view illustrating step 3 of a method in accordance with the principles of the present disclosure;

FIG. 3D is a side, cross-sectional view illustrating step 4 of a method in accordance with the principles of the present disclosure;

FIG. 3E is a side, cross-sectional view illustrating step 5 of a method in accordance with the principles of the present disclosure;

FIG. 3F is a side, cross-sectional view illustrating step 6 of a method in accordance with the principles of the present disclosure;

FIG. 4A is a side, cross-sectional view illustrating step 1 of a method in accordance with the principles of the present disclosure;

FIG. 4B is a side, cross-sectional view illustrating step 2 of a method in accordance with the principles of the present disclosure;

FIG. 40 is a side, cross-sectional view illustrating step 3 of a method in accordance with the principles of the present disclosure;

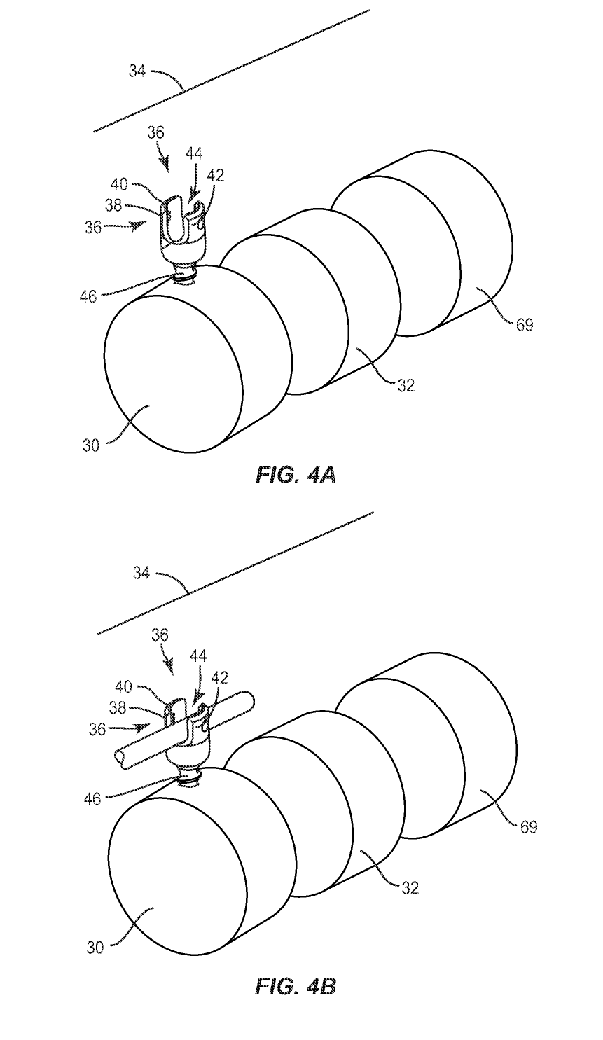

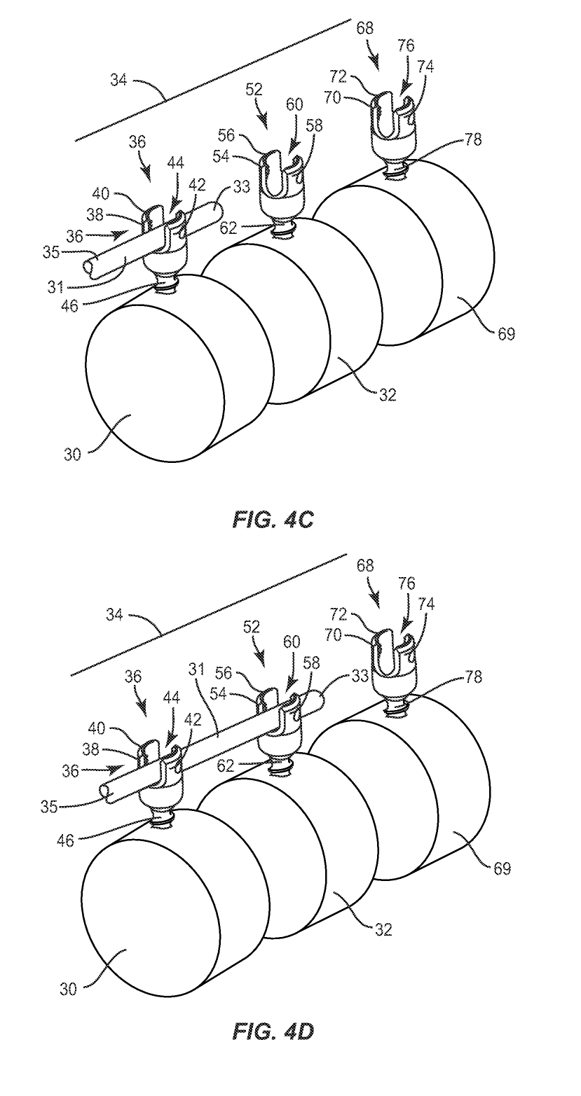

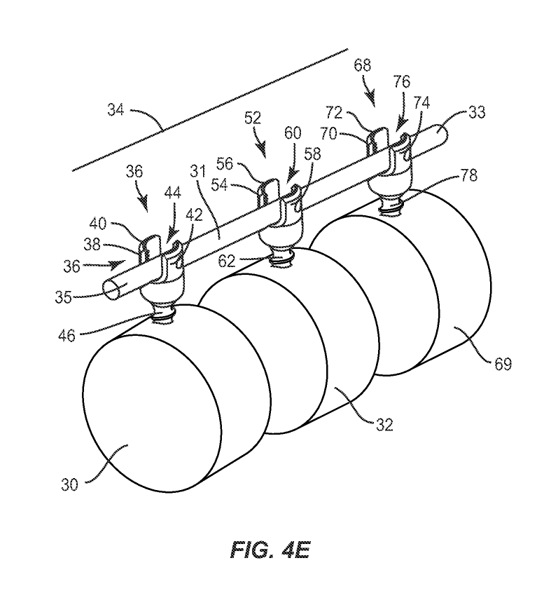

FIG. 4D is a side, cross-sectional view illustrating step 4 of a method in accordance with the principles of the present disclosure; and

FIG. 4E is a side, cross-sectional view illustrating step 5 of a method in accordance with the principles of the present disclosure.

Like reference numerals indicate similar parts throughout the figures.

### DETAILED DESCRIPTION OF THE INVENTION

The exemplary embodiments of the methods of use disclosed are discussed in terms of methods for the treatment of musculoskeletal disorders and more particularly, in terms of methods for installing a vertebral construct. It is envisioned that methods disclosed may reduce the recovery time of a patient after surgery by splitting, but not cutting, muscle fibers adjacent one or more vertebral body as a vertebral construct is anchored to two or more bone fixation apparatuses. It is further envisioned that the methods disclosed could include providing extenders, reducers and translators, which can be used to install a vertebral construct, such as a rod, to a bone fastener, such as a bone anchor or bone screw.

The disclosed methods may be employed, for example, with an open or mini-open surgical technique to deliver and introduce a vertebral construct to a bone fastener at a surgical site within a body of a patient, for example, a section of a spine. In one embodiment, the components used with the disclosed methods are configured to position a vertebral construct into engagement with two or more bone fasteners for a correction treatment to treat various spine pathologies.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed methods may be alternatively employed in a surgical treatment with a patient in a prone or supine position, and/or employ various surgical approaches to the spine, including anterior, posterior, posterior mid-line, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The components used in the disclosed methods can be fabricated from biologically acceptable materials suitable for medical applications, including metals, synthetic polymers, ceramics and bone material and/or their composites, depending on the particular application and/or preference of a medical practitioner. For example, the components used in the disclosed methods, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyimide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components used in the disclosed methods may have material composites, including the above materials, to achieve various desired characteristics such as strength, rigidity, elasticity, compliance, biomechanical performance, durability and radiolucency or imaging preference. The components used in the disclosed methods, individually or collectively, may also be fabricated from a heterogeneous material such as a combination of two or more of the above-described materials. The components used in the disclosed methods may be monolithically formed, integrally connected or include fastening elements and/or instruments, as described herein.

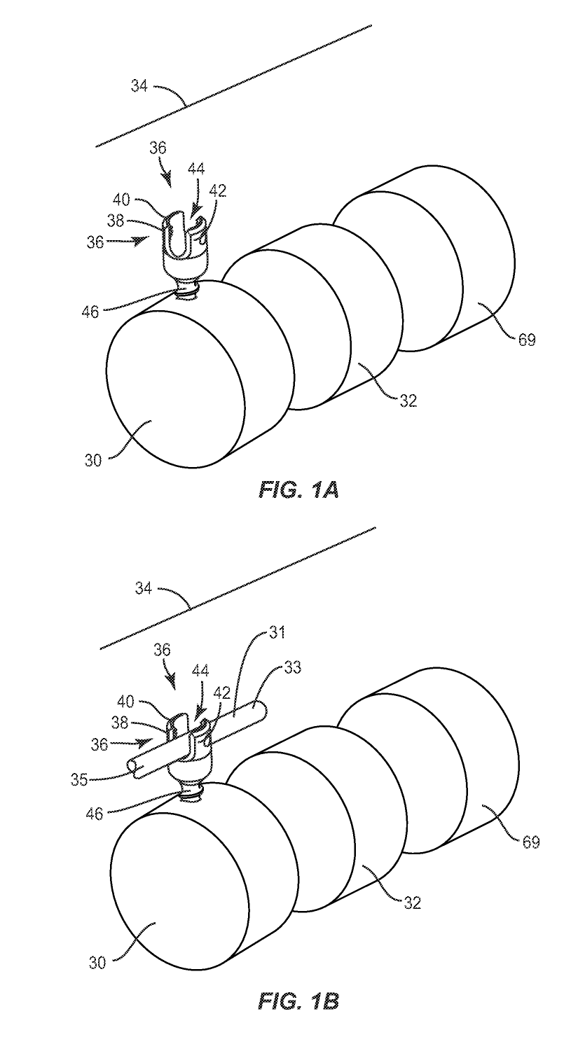

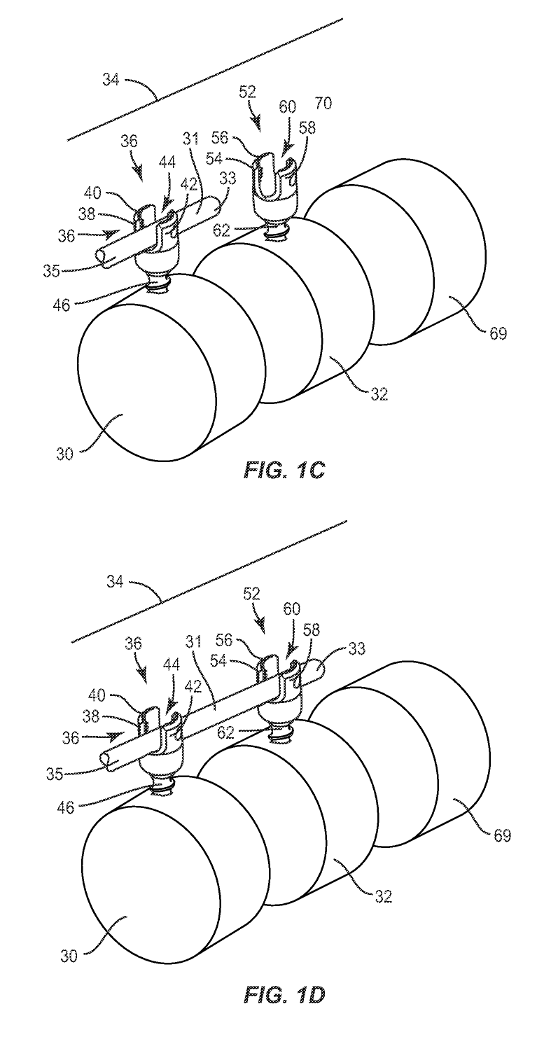

The following discussion includes a description of methods for installing a vertebral construct in accordance with the principles of the present disclosure. Alternate embodiments are also disclosed. Reference will now be made in detail to the exemplary embodiments of the present disclosure, which are illustrated in the accompanying figures. Turning now to FIGS. 1A-1D, there is illustrated steps 1-4 of a method for installing a vertebral construct in accordance with the principles of the present disclosure.

In use, to treat the affected section of the spine, a medical practitioner obtains access to a surgical site including a first vertebral body 30 and a second vertebral body 32 in any appropriate manner, such as, for example, through one or more incision extending through skin 34 of a patient and retraction of tissues in an open or mini-open surgical technique. Once access to the surgical site is obtained, the particular surgical procedure is performed for treating the spinal disorder. During surgical treatment, one or more vertebral constructs, such as, for example, a vertebral rod 31 may be attached via one or more bone fixation apparatus to the exterior of first vertebral body 30 and/or second vertebral body 32. It is envisioned that the vertebral constructs include vertebral rods having dynamic or flexible characteristics or have dynamic or flexible components. The vertebral rods can have varying geometries and lengths as well.

Step 1 of one embodiment of the disclosed method, shown in FIG. 1A, involves anchoring a first bone fixation apparatus 36 having a receiver 38 defined by a pair of opposing arms 40, 42 defining a channel 44 therebetween configured to receive the vertebral rod 31 and a bone anchor having 46 a lower portion configured for engaging the first vertebral body 30 and a head engaged with a lower surface of receiver 38 into first vertebral body 30. In one embodiment, channel 44 has a generally U-shaped cross-section configured to receive and couple a vertebral construct having a cylindrical cross-section. However, it is envisioned that channel 44 may have other cross-sectional configurations, including, for example, V-shaped, W-shaped, polygonal or tapered.

Once first bone fixation apparatus 30 is anchored in first vertebral body 30, vertebral rod 31 is positioned within channel 44. That is, vertebral rod 31 is inserted into channel 44 such that vertebral rod 31 is seated within channel 44, as shown in FIG. 1B. Next, a second bone fixation apparatus 52 having a receiver 54 defined by a pair of opposing arms 56, 58 defining a channel 60 therebetween configured to receive vertebral rod 31 and a bone anchor 62 having a lower portion configured for engaging second vertebral body 32 and a head engaged with a lower surface of receiver 54 is anchored into second vertebral body 32, as shown in FIG. 1C.

Vertebral rod 31 extends between a proximal end 33 and a distal end 35. After second bone fixation apparatus 52 is anchored into second vertebral body 32, proximal end 33 of vertebral rod 31 is advanced proximally toward second bone fixation apparatus 52, while maintaining at least a portion of vertebral rod 31 within channel 44 of first bone fixation apparatus 36, until at least a portion of vertebral rod 31 is received within channel 60 of second bone fixation apparatus 52, at which point, vertebral rod 31 is seated within channels 44, 60 of first and second bone fixation apparatuses 36, 52 so as to secure vertebral rod 31 to first and second bone fixation apparatuses 36, 52. That is, proximal end 33 of vertebral rod is manipulated sub-dermally from a location proximate first bone fixation apparatus 36 to a location proximate second bone fixation apparatus 52 such that at least a portion of 33 of vertebral rod 31 is received within channel 60 of second bone fixation apparatus 52, as shown in FIG. 10. As vertebral rod 31 is advanced from first bone fixation apparatus 36 to second bone fixation apparatus 52, vertebral rod 31 may advance through muscle(s) adjacent to first and second bone fixation apparatuses 36, 52 such that vertebral rod 31 splits the fibers of such muscle(s) longitudinally without cutting the fibers of the muscle(s).

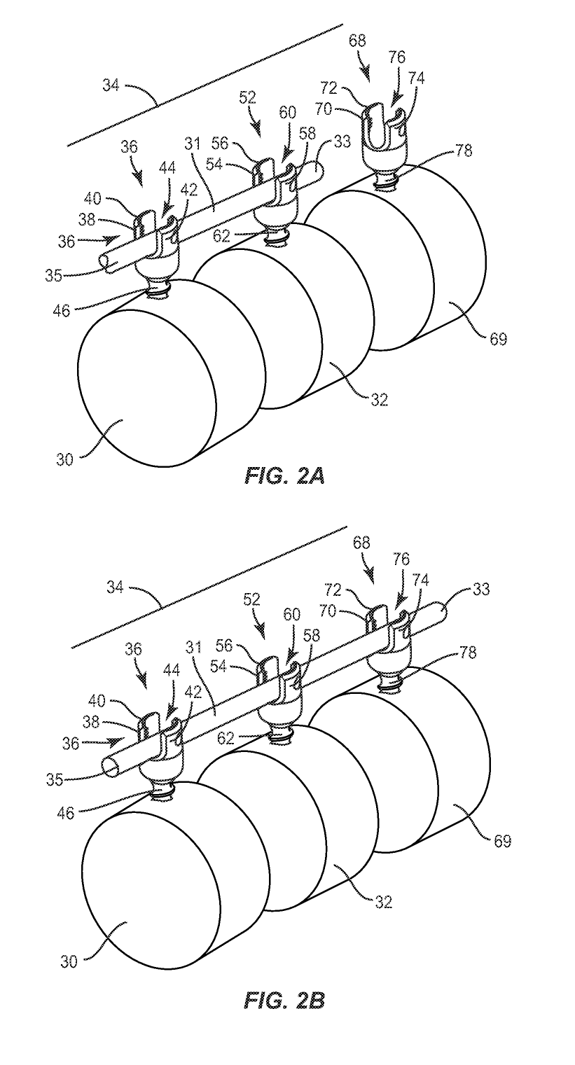

In one embodiment, shown in FIGS. 2A and 2B, a method is provided in accordance with the principles of the present disclosure in which a third bone fixation apparatus 68 is anchored into a third vertebral body 69, adjacent vertebral body 32, and vertebral 31 has a length sufficient to extend between first, second and third bone fixation apparatuses 36, 52,68, such that vertebral rod 31 may be positioned within third bone fixation apparatus 68, as shown in FIG. 2A, after vertebral rod 31 is positioned within first and second bone fixation apparatuses 36, 52, as shown in FIG. 1D. In particular, as shown in FIG. 2A, third bone fixation apparatus 68 includes a receiver 70 defined by a pair of opposing arms 72, 74 defining a channel 76 therebetween configured to receive vertebral rod 31 and a bone anchor 78 having a lower portion configured for engaging third vertebral body 69 and a head engaged with a lower surface of receiver 70 is anchored into third vertebral body 69. In one embodiment, first bone fixation apparatus 36 and/or second bone fixation apparatus 52 is/are a multi-axial or poly-axial bone screw, such as the multi-axial bone anchor apparatus disclosed in U.S. Pat. No. 5,797,911 to Sherman et al., or the multi-axial bone anchor apparatus disclosed in U.S. Pat. No. 6,280,442 to Barker, et al., both of which are incorporated herein by reference in their entirely.

Next, proximal end 33 of vertebral rod 31 is advanced proximally toward third bone fixation apparatus 68, while maintaining at least a portion of vertebral rod 31 within channels 44, 60 of first and second bone fixation apparatuses 36, 52, until at least a portion of vertebral rod 31 is received within channel 76 of third bone fixation apparatus 68, at which point, vertebral rod 31 is seated within channels 44, 60, 76 of first, second and third bone fixation apparatuses 36, 52, 68 so as to secure vertebral rod 31 to first, second and third bone fixation apparatuses 36, 52, 68. That is, proximal end 33 of vertebral rod is manipulated sub-dermally from a location proximate second bone fixation apparatus 52 to a location proximate third bone fixation apparatus 68 such at least a portion of vertebral rod 31 is received within channel 76 of third bone fixation apparatus 68, as shown in FIG. 2B. It is understood that as vertebral rod 31 is advanced from second bone fixation apparatus 52 to third bone fixation apparatus 68, vertebral rod 31 may advance through muscle(s) adjacent second and third bone fixation apparatuses 52, 68 such that vertebral rod 31 splits the fibers of such muscle(s) longitudinally without culling the fibers of the muscle(s).

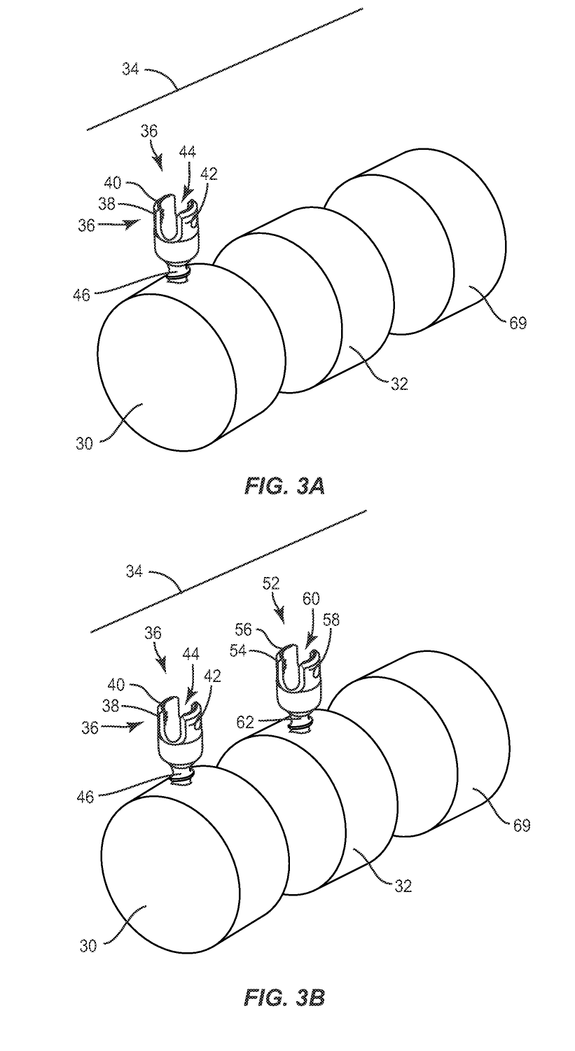

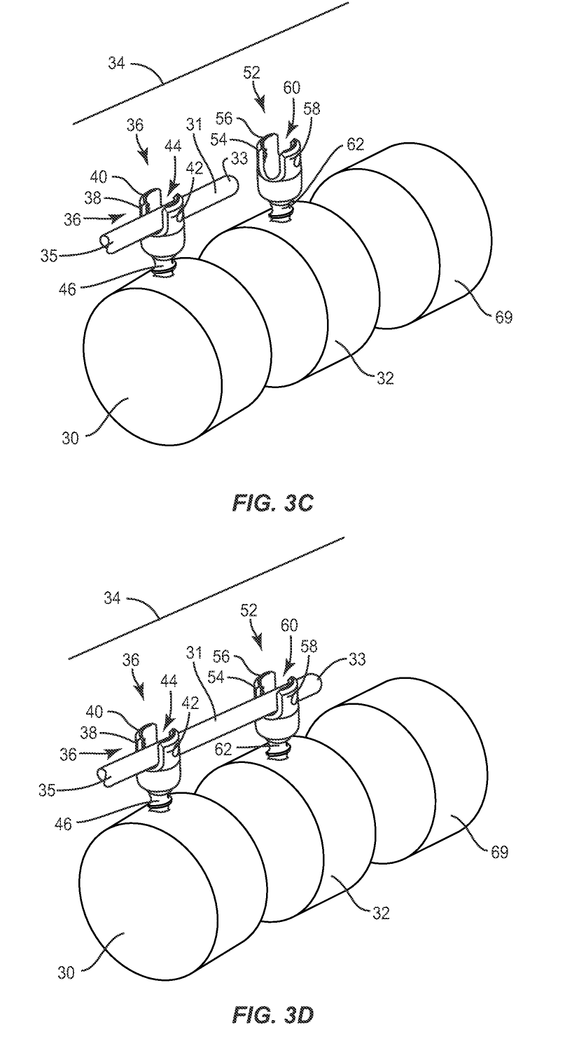

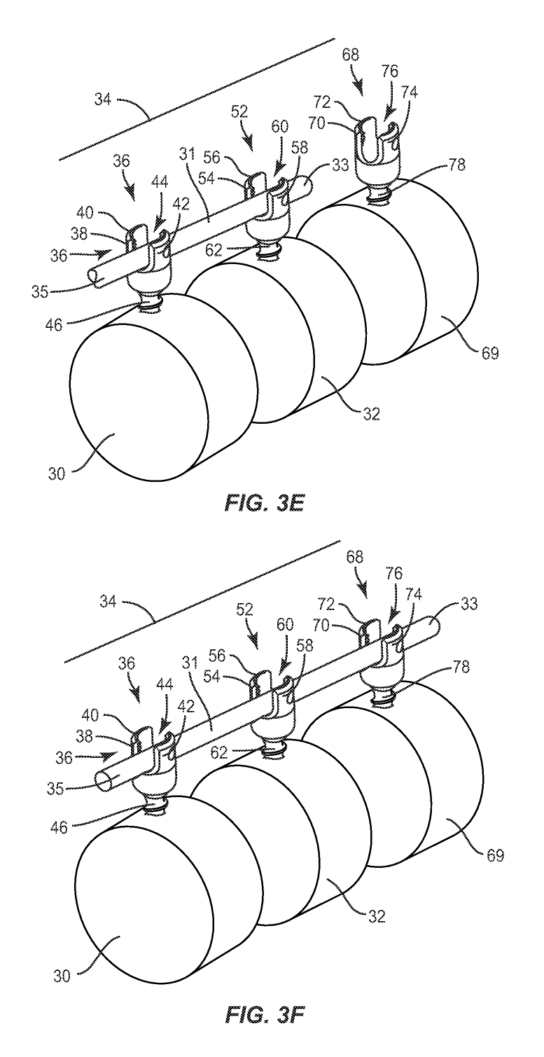

In one embodiment, shown in FIGS. 3A-3F, a method for installing a vertebral construct is provided in which vertebral rod 31 is positioned within first and second bone fixation apparatuses 36, 52, and is then advanced such that vertebral rod 31 is positioned within third bone fixation apparatus 68. In particular, first bone fixation apparatus 36 is anchored into first vertebral body 30, as shown in FIG. 3A. Second bone fixation apparatus 52 is then anchored into second vertebral body 32, as shown in FIG. 3B. After second bone fixation apparatus 52 is anchored into second vertebral body 32, vertebral rod 31 is positioned within channel 44 of first bone fixation apparatus 36. That is, vertebral rod 31 is inserted into channel 44 such that vertebral rod 31 is seated within channel 44, as shown in FIG. 3C.

Proximal end 33 of vertebral rod 31 is advanced proximally toward second bone fixation apparatus 52, while maintaining at least a portion of vertebral rod 31 within channel 44 of first bone fixation apparatus 36, until at least a portion of vertebral rod 31 is received within channel 60 of second bone fixation apparatus 52, at which point, vertebral rod 31 is seated within channels 44, 60 of first and second bone fixation apparatuses 36, 52 so as to secure vertebral rod 31 to first and second bone fixation apparatuses 36, 52. That is, proximal end 33 of vertebral rod is manipulated sub-dermally from a location proximate first bone fixation apparatus 36 to a location proximate second bone fixation apparatus 52 such that at least a portion of 33 of vertebral rod 31 is received within channel 60 of second bone fixation apparatus 52, as shown in FIG. 3D.

Next, third bone fixation apparatus 68 is anchored into third vertebral body 69, as show in FIG. 3E. Proximal end 33 of vertebral rod 31 is then advanced proximally toward third bone fixation apparatus 68, while maintaining at least a portion of vertebral rod 31 within channels 44, 60 of first and second bone fixation apparatuses 36, 52, until at least a portion of vertebral rod 31 is received within channel 76 of third bone fixation apparatus 68, at which point, vertebral rod 31 is seated within channels 44, 60, 76 of first, second and third bone fixation apparatuses 36, 52, 68 so as to secure vertebral rod 31 to first, second and third bone fixation apparatuses 36, 52, 68. That is, proximal end 33 of vertebral rod is manipulated sub-dermally from a location proximate second bone fixation apparatus 52 to a location proximate third bone fixation apparatus 68 such at least a portion of vertebral rod 31 is received within channel 76 of third bone fixation apparatus 68, as shown in FIG. 3F.

In one embodiment, shown in FIGS. 4A-4E, a method for installing a vertebral construct is provided in which vertebral rod 31 is first positioned within first bone fixation apparatuses 36, is then advanced such that vertebral rod 31 is positioned within second bone fixation apparatus 52, and is then further advanced such that vertebral rod 31 is positioned within third bone fixation apparatus 68. In particular, first bone fixation apparatus 36 is anchored into first vertebral body 30, as shown in FIG. 4A. Once first bone fixation apparatus 30 is anchored in first vertebral body 30, vertebral rod 31 is positioned within channel 44. That is, vertebral rod 31 is inserted into channel 44 such that vertebral rod 31 is seated within channel 44, as shown in FIG. 4B. Following Step 2, second bone fixation apparatus 52 is anchored into second vertebral body 32 and third bone fixation apparatus 68 is anchored into third vertebral body 69, as shown in FIG. 4C.

Next, proximal end 33 of vertebral rod 31 is advanced proximally toward second bone fixation apparatus 52, while maintaining at least a portion of vertebral rod 31 within channel 44 of first bone fixation apparatus 36, until at least a portion of vertebral rod 31 is received within channel 60 of second bone fixation apparatus 52, at which point, vertebral rod 31 is seated within channels 44, 60 of first and second bone fixation apparatuses 36, 52 so as to secure vertebral rod 31 to first and second bone fixation apparatuses 36, 52. That is, proximal end 33 of vertebral rod is manipulated sub-dermally from a location proximate first bone fixation apparatus 36 to a location proximate second bone fixation apparatus 52 such that at least a portion of 33 of vertebral rod 31 is received within channel 60 of second bone fixation apparatus 52, as shown in FIG. 4D.

Next, proximal end 33 of vertebral rod 31 is advanced proximally toward third bone fixation apparatus 68, while maintaining at least a portion of vertebral rod 31 within channels 44, 60 of first and second bone fixation apparatuses 36, 52, until at least a portion of vertebral rod 31 is received within channel 76 of third bone fixation apparatus 68, at which point, vertebral rod 31 is seated within channels 44, 60, 76 of first, second and third bone fixation apparatuses 36, 52, 68 so as to secure vertebral rod 31 to first, second and third bone fixation apparatuses 36, 52, 68. That is, proximal end 33 of vertebral rod is manipulated sub-dermally from a location proximate second bone fixation apparatus 52 to a location proximate third bone fixation apparatus 68 such at least a portion of vertebral rod 31 is received within channel 76 of third bone fixation apparatus 68, as shown in FIG. 4E.

In one embodiment, first vertebral body 30 is a first vertebra, second vertebral body 32 is a second vertebra adjacent the first vertebra, and the third vertebral body 69 is a third vertebra adjacent the second vertebra. As such, the disclosed method may be used to install a vertebral construct, such as, for example, vertebral rod 31 via one or more bone fasteners, such as, for example, first, second and third bone fixation apparatuses 36, 52, 68, wherein first, second and third bone fixation apparatuses 36, 52, 68 are each anchored to a different vertebra. Alternatively, first and second vertebral bodies 30, 32 may define a first vertebra and the third vertebral body 69 is a second vertebra adjacent the first vertebra. That is, the disclosed method may be used to attach a vertebral construct, such as, for example, vertebral rod 31 via bone fasteners, such as, for example, first and second bone fixation apparatuses 36, 52 to a first vertebra and then attach the vertebral construct to a vertebra adjacent the first vertebra via a bone fastener, such as, for example, third bone fixation apparatus 68. In one embodiment, first and second bone fixation apparatuses 36, 52 may be opposite endplates of the same vertebra. For example, first vertebral body 30 may be an upper endplate of a vertebra and second vertebral body 32 may be a lower endplate of the same vertebra.

It is envisioned that the disclosed method may include anchoring one or more additional bone fixation apparatuses, having a configuration similar to first, second and third bone fixation apparatuses 36, 52, 68, in one or more additional vertebral bodies and advancing vertebral rod 31 proximally toward the one or more additional bone fixation apparatuses, while maintaining at least a portion of vertebral rod 31 within channels 44, 60, 76 of first, second and third bone fixation apparatuses 36, 52, 68, until at least a portion of vertebral rod 31 is received within a channel of a receiver of the one or more additional bone fixation apparatuses.

In one embodiment, a vertebral construct, such as, for example, vertebral rod 31 may be reduced into receivers 38, 54, 70 of the first, second and third bone fixation apparatuses 36, 52, 68 to bring the vertebral construct to final position within receivers 38, 54, 70. It is envisioned that a tool, such as, for example, a reducer may be used to reduce the vertebral construct into receivers 38, 54, 70. It is further envisioned that the vertebral construct may be reduced into receivers 38, 54, 70 after the vertebral construct is positioned into receivers 38, 54, 70. Alternatively, the vertebral construct may be reduced into receivers 38, 54, 70 sequentially. That is, the vertebral construct may be reduced into receiver 38 after the vertebral construct is positioned therein, then the vertebral construct may be reduced into receiver 54 after the vertebral construct is positioned therein, etc. After the vertebral construct is reduced into receivers 38, 54, 70, the vertebral construct may be secured within receivers 38, 54, 70 via set screws. It is envisioned that receivers 38, 54, 70 may have threads on the inner surfaces thereof configured to mate with threads of the set screws such that the set screws can be threaded into receivers 38, 54, 70.

In one embodiment, an instrument, such as, for example, an extender may engage an upper surface of receivers 38, 54, 70 of first, second and third bone fixation apparatuses 36, 52, 68 having a first wall and a second wall defining an implant cavity therebetween in communication with channels 44, 60, 76 of receivers 38, 54, 70 thereby permitting a vertebral construct, such as, for example, vertebral rod 31 to be advanced from a proximal end of each implant cavity to a distal end of the implant cavity and into channels 44, 60, 76. It is envisioned that the extenders may have a length such that the implant cavities of each extender are accessible outside the body of a patient when first, second and third bone fixation apparatuses 36, 52, 68 are fully implanted in a vertebral body.

In one embodiment, vertebral rod 31 may be shaped by, for example, bending vertebral rod 31 into a desired configuration based on the relative positions of first, second and third bone fixation apparatuses 36, 52, 68 so as to define a particular curvature of a final configuration of vertebral rod 31. It is envisioned that vertebral rod 31 may be shaped such that vertebral rod 31 substantially conforms with the curvature of a spine of a patient. Vertebral rod 31 may be shaped in situ or outside the body of a patient.

It is envisioned that first, second and/or third bone fixation apparatuses 36, 52, 68 may be coated with agents, such as, for example, antibiotics, anti-inflammatory drugs, therapeutic proteins, therapeutic peptides, therapeutic nucleic acids (as naked plasmid or a component of an integrating or non-integrating gene therapy vector system), analgesics and anesthetics. It is further envisioned that first, second and/or third bone fixation apparatuses 36, 52, 68 may be coated with therapeutic polynucleotides, polypeptides, biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines.

It is envisioned that the agent may include one or a plurality of therapeutic agents and/or pharmacological agents for release, including sustained release, to treat, for example, pain, inflammation and degeneration. The agents may include pharmacological agents, such as, for example, antibiotics, anti-inflammatory drugs including but not limited to steroids, anti-viral and anti-retroviral compounds, therapeutic proteins or peptides; therapeutic nucleic acids (as naked plasmid or a component of an integrating or non-integrating gene therapy vector system), and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids; opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

First, second and third bone fixation apparatuses 36, 52, 68 can be made of radiolucent materials such as polymers. Radiomarkers may be included for identification under x-ray, fluoroscopy, CT or other imaging techniques. It is envisioned that the use of microsurgical and image guided technologies may be employed to access, view and repair spinal deterioration or damage.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for installing a vertebral construct, the method comprising the steps of:
anchoring a first bone fixation apparatus having a receiver defined by a pair of opposing arms defining a channel therebetween configured to receive the vertebral construct and a bone anchor having a lower portion configured for engaging a first vertebral body and a head engaged with a lower surface of the receiver into the first vertebral body;
positioning the vertebral construct within the channel of the receiver of the first bone fixation apparatus;
anchoring a second bone fixation apparatus having a receiver defined by a pair of opposing arms defining a channel therebetween configured to receive the vertebral construct and a bone anchor having a lower portion configured for engaging a second vertebral body and a head engaged with a lower surface of the receiver into the second vertebral body; and
positioning the vertebral construct positioned within the channel of the receiver of the first bone fixation apparatus within the channel of the receiver of the second bone fixation apparatus so as to secure the vertebral construct to the first and second bone fixation apparatuses.

2. The method of claim 1, further comprising:
anchoring a third bone fixation apparatus having a receiver defined by a pair of opposing arms defining a channel therebetween configured to receive the vertebral construct and a bone anchor having a lower portion configured for engaging a third vertebral body and a head engaged with a lower surface of the receiver into the third vertebral body; and
positioning the vertebral construct positioned within the channels of the receivers of the first and second bone fixation apparatuses within the channel of the receiver of the third bone fixation apparatus so as to secure the vertebral construct to the first, second and third bone fixation apparatuses.

3. The method of claim 2, wherein the vertebral construct is a vertebral rod.

4. The method of claim 3, further comprising:
bending the rod into a desired configuration based on the relative positions of the first, second and third bone fixation apparatuses so as to define a particular curvature of a final configuration of the vertebral construct.

5. The method of claim 2, further comprising:
manipulating an end of the vertebral construct sub-dermally from a location proximate the first bone fixation apparatus to a location proximate the second bone fixation apparatus; and
manipulating an end of the vertebral construct sub-dermally from a location proximate the second bone fixation apparatus to a location proximate the third bone fixation apparatus.

6. The method of claim 2, wherein the first vertebral body is a first vertebra, the second vertebral body is a second vertebra adjacent the first vertebra, and the third vertebral body is a third vertebra adjacent the second vertebra.

7. The method of claim 2, wherein the first and second vertebral bodies define a first vertebra and the third vertebral body is a second vertebra adjacent the first vertebra.

8. The method of claim 2, wherein the first, second and third bone fixation apparatuses each further comprise:
an extender engaged with an upper surface of the receiver having a first wall and a second wall defining an implant cavity therebetween in communication with the channel of the receiver;
wherein the vertebral construct is advanced from a proximal end of the implant cavity to a distal end of the implant cavity and into the channel.

9. The method of claim 2, further comprising:
reducing the vertebral construct into the receivers of the first, second and third bone fixation apparatuses to a final position; and
securing the vertebral construct within the receivers of the first, second and third bone fixation apparatuses with a set screw which is inserted into the receivers of the first, second and third bone fixation apparatuses.

10. The method of claim 2, wherein at least one of the first, second and third bone fixation apparatuses is coated with an agent selected from the group consisting of antibiotics, anti-inflammatory drugs, therapeutic proteins, therapeutic peptides, therapeutic nucleic acids (as naked plasmid or a component of an integrating or non-integrating gene therapy vector system), analgesics and anesthetics.

11. A method for installing a vertebral construct, the method comprising the steps of:
anchoring a first bone fixation apparatus having a receiver defined by a pair of opposing arms defining a channel therebetween configured to receive the vertebral construct and a bone anchor having a lower portion configured for engaging a first vertebral body and a head engaged with a lower surface of the receiver into the first vertebral body;
anchoring a second bone fixation apparatus having a receiver defined by a pair of opposing arms defining a channel therebetween configured to receive the vertebral construct and a bone anchor having a lower portion configured for engaging a second vertebral body and a head engaged with a lower surface of the receiver into the second vertebral body;
positioning the vertebral construct within the channel of the receiver of the first bone fixation apparatus;
positioning the vertebral construct positioned within the receiver of the channel of the first bone fixation apparatus within the channel of the receiver of the second bone fixation apparatus;
anchoring a third bone fixation apparatus having a receiver defined by a pair of opposing arms defining a channel therebetween configured to receive the vertebral construct and a bone anchor having a lower portion configured for engaging a third vertebral body and a head engaged with a lower surface of the receiver into the third vertebral body; and
positioning the vertebral construct positioned within the receivers of the first and second bone fixation apparatuses within the channel of the receiver of the third bone fixation apparatus so as to secure the vertebral construct to the first, second and third bone fixation apparatuses.

12. The method of claim 11, wherein the vertebral construct is a vertebral rod.

13. The method of claim 12, further comprising:
bending the rod into a desired configuration based on the relative positions of the first, second and third bone fixation apparatuses so as to define a particular curvature of a final configuration of the vertebral construct.

14. The method of claim 11, further comprising:
manipulating an end of the vertebral construct sub-dermally from a location proximate the first bone fixation apparatus to a location proximate the second bone fixation apparatus; and
manipulating an end of the vertebral construct sub-dermally from a location proximate the second bone fixation apparatus to a location proximate the third bone fixation apparatus.

15. The method of claim 11, further comprising:
reducing the vertebral construct into the receivers of the first, second and third bone fixation apparatuses to a final position; and
securing the vertebral construct within the receivers of the first, second and third bone fixation apparatuses with a set screw which is inserted into the receivers of the first, second and third bone fixation apparatuses.

16. A method for installing a vertebral construct, the method comprising the steps of:
anchoring a first bone fixation apparatus having a receiver defined by a pair of opposing arms defining a channel therebetween configured to receive the vertebral construct and a bone anchor having a lower portion configured for engaging a first vertebral body and a head engaged with a lower surface of the receiver into the first vertebral body;
positioning the vertebral construct within the channel of the receiver of the first bone fixation apparatus;
anchoring a second bone fixation apparatus having a receiver defined by a pair of opposing arms defining a channel therebetween configured to receive the vertebral construct and a bone anchor having a lower portion configured for engaging a second vertebral body and a head engaged with a lower surface of the receiver into the second vertebral body;
anchoring a third bone fixation apparatus having a receiver defined by a pair of opposing arms defining a channel therebetween configured to receive the vertebral construct and a bone anchor having a lower portion configured for engaging a third vertebral body and a head engaged with a lower surface of the receiver into the third vertebral body;
positioning the vertebral construct positioned within the channel of the receiver of the first bone fixation apparatus within the channel of the receiver of the second bone fixation apparatus; and
positioning the vertebral construct positioned within the channels of receivers of the first and second bone fixation apparatuses within the channel of the receiver of the third bone fixation apparatus so as to secure the vertebral construct to the first, second and third bone fixation apparatuses.

17. The method of claim 11, wherein the vertebral construct is a vertebral rod.

18. The method of claim 17, further comprising:
bending the rod into a desired configuration based on the relative positions of the first, second and third bone fixation apparatuses so as to define a particular curvature of a final configuration of the vertebral construct.

19. The method of claim 16, further comprising:
manipulating an end of the vertebral construct sub-dermally from a location proximate the first bone fixation apparatus to a location proximate the second bone fixation apparatus; and
manipulating an end of the vertebral construct sub-dermally from a location proximate the second bone fixation apparatus to a location proximate the third bone fixation apparatus.

20. The method of claim 16, further comprising:
reducing the vertebral construct into the receivers of the first, second and third bone fixation apparatuses to a final position; and
securing the vertebral construct within the receivers of the first, second and third bone fixation apparatuses with a set screw which is inserted into the receivers of the first, second and third bone fixation apparatuses.
